(19) United States
(12) Patent Application Publication
(10) Pub. No.: US 2026/0185913 A1
(43) Pub. Date: Jul. 2, 2026

(54) Systems and Methods for Measuring Osmotic Pressure

(71) Applicant: The Regents of the University of California, Oakland, CA (US)

(72) Inventors: Antoine Vian, Santa Barbara, CA (US); Otger Campas, Dresden (DE)

(73) Assignee: The Regents of the University of California, Oakland, CA (US)

(21) Appl. No.: 19/130,055

(22) PCT Filed: Nov. 1, 2023
(86) PCT No.: PCT/US2023/078408

§ 371 Date: May 14, 2025

Related U.S. Application Data
(60) Provisional application No. 63/383,647, filed on Nov. 14, 2022.

(51) Int. Cl. G01N 13/04; G01N 13/00; G01N 33/483

(52) U.S. Cl. CPC G01N 13/04; G01N 33/4833; G01N 2013/003

(57) ABSTRACT

Systems and methods for measuring osmotic pressure are described. In many embodiments, double emulsion droplets can be used as osmotic pressure sensors to measure local osmotic pressure. The double emulsion droplets can measure osmotic pressure in living cells and/or tissues.

### CROSS-REFERENCE TO RELATED APPLICATIONS

The current application claims the benefit and priority of U.S. Provisional Patent Application No. 63/383,647 entitled “Systems and Methods for Measuring Osmotic Pressure” filed Nov. 14, 2022. The disclosure of U.S. Provisional Patent Application No. 63/383,647 is incorporated by reference in its entirety for all purposes.

### STATEMENT OF FEDERALLY SPONSORED RESEARCH

This invention was made with government support under R01 GM135380 awarded by the National Institutes of Health. The government has certain rights in the invention.

### FIELD OF THE INVENTION

The current disclosure is directed to systems and methods for measuring osmotic pressure; and more particularly to systems and methods for direct in vivo and in situ measurements of osmotic pressure in living cells and tissues.

### BACKGROUND

Mechanics is known to play a fundamental role in many cellular and developmental processes. Beyond active forces and material properties, osmotic pressure (or osmolarity) is believed to control essential characteristics of cell, tissues and organs. However, it remains very challenging to perform in situ and in vivo measurements of osmotic pressure.

### BRIEF SUMMARY

Systems and methods in accordance with various embodiments of the invention implement double emulsion droplet as sensors for osmotic pressure measurement. The double emulsion droplet sensors in accordance with several embodiments enable local measurements of osmotic pressure intra-cellularly and extra-cellularly within 3D multicellular systems including (but not limited to) developing embryos, living tissues, tissue explants, organoids, multicellular aggregates, cells, and living cells. The double emulsion droplets can be inserted in cellular environments. The double emulsion microdroplets in accordance with some embodiments can be an oil droplet containing a smaller aqueous droplet. The aqueous droplet can contain a calibrated concentration of osmolyte in accordance with various embodiments. In certain embodiments, the oil surrounding the inner aqueous droplet can act as a protective shell while allowing water transport, effectively behaving as a water permeable layer. The double emulsion droplets can shrink (or expand) in size as water transports outwards from (or inwards into) the droplets. The change in the volume of the double emulsion droplets can be corelated with the osmotic pressure. Several embodiments measure and calibrate the size of the droplets as the pressure changes and establish a reference (calibrated double emulsion droplets). Many embodiments measure the osmotic pressure by monitoring the size of droplets. Several embodiments control various parameters including (but not limited to) the osmolyte concentration in the inner droplet, the surfactants in the oil, and the relative inner/outer droplet sizes, to generate osmotic pressure sensors with well-defined characteristics. In this disclosure, osmotic pressure, or osmolarity, or osmotic potential can be used interchangeably, unless specifically stated otherwise.

Some embodiments include an osmotic pressure sensor comprising: a double emulsion droplet comprising: an oil droplet with a first diameter; wherein the oil droplet has an inner surface and an outer surface and the inner and the outer surfaces are modified with at least one surfactant to facilitate water transport through the oil droplet; and at least one aqueous droplet enclosed in the oil droplet; wherein the at least one aqueous droplet has a second diameter less than the first diameter; wherein the at least one aqueous droplet comprises an inner osmolyte and an environment outside the double emulsion droplet comprises an outer osmolyte; wherein a concentration difference between the outer osmolyte and the inner osmolyte drives water transport through the oil droplet such that the double emulsion droplet either shrinks or expands in volume; and wherein a volume change in the double emulsion droplet measures the osmotic pressure.

In some embodiments, the first diameter is less than or equal to 160 microns.

In some embodiments, the inner osmolyte has a molecular weight of less than 6,000 Dalton.

In some embodiments, the oil droplet comprises an oil selected from the group consisting of: a fluorocarbon oil, a silicone oil, and a biocompatible oil.

In some embodiments, the surfactant comprises a molecular component with at least one hydrophilic part and at least one hydrophobic part, lipids, sodium dodecyl sulfate, sodium lauryl sulfate, poly(N-isopropylacrylamide), or a fluorinated surfactant.

In some embodiments, the inner osmolyte comprises polyethylene glycol or Dextran; the oil droplet comprises a fluorocarbon oil, a silicone oil, or a biocompatible oil; and the at least one surfactant comprises Krytox™-PEG (600), Krytox™, or perfluoro-15-crown-5-ether.

In some embodiments, the at least one aqueous droplet further comprises a color dye or a fluorescent color dye.

In some embodiments, the oil droplet further comprises a color dye or a fluorescent color dye.

In some embodiments, the osmotic pressure sensor is configured to measure the osmotic pressure in vitro, in vivo or in situ.

Some embodiments include a method for measuring an osmotic pressure, comprising: obtaining a double emulsion droplet, wherein the droplet comprises: an oil droplet with a first diameter; wherein the oil droplet has an inner surface and an outer surface and the inner and the outer surfaces are modified with at least one surfactant to facilitate water transport through the oil droplet; and at least one aqueous droplet enclosed in the oil droplet; wherein the at least one aqueous droplet has a second diameter less than the first diameter; wherein the at least one aqueous droplet comprises an inner osmolyte; and wherein an osmolyte concentration difference drives water transport through the oil droplet such that the double emulsion droplet either shrinks or expands in volume; calibrating a relationship between a volume change of the double emulsion droplet and an external osmotic pressure in a reference solution; placing the double emulsion droplet in an environment comprising an outer osmolyte; monitoring a volume change of the double emulsion droplet in the environment; and obtaining the osmotic pressure of the environment using the calibrated relationship.

Some embodiments further comprise using a microscope to monitor the volume change of the double emulsion droplet in the environment.

Some embodiments further comprise placing the double emulsion droplet in the environment using a microneedle.

In some embodiments, the first diameter is less than or equal to 160 microns.

In some embodiments, the inner osmolyte has a molecular weight of less than 6,000 Dalton.

In some embodiments, the oil droplet comprises an oil selected from the group consisting of: a fluorocarbon oil, a silicone oil, and a biocompatible oil.

In some embodiments, the surfactant comprises a molecular component with at least one hydrophilic part and at least one hydrophobic part, lipids, sodium dodecyl sulfate, sodium lauryl sulfate, poly(N-isopropylacrylamide), or a fluorinated surfactant.

In some embodiments, the inner osmolyte comprises polyethylene glycol or Dextran; the oil droplet comprises a fluorocarbon oil, a silicone oil, and a biocompatible oil; and the at least one surfactant comprises Krytox™-PEG (600), Krytox™, or perfluoro-15-crown-5-ether.

In some embodiments, the at least one aqueous droplet further comprises a color dye or a fluorescent color dye.

In some embodiments, the oil droplet further comprises a color dye or a fluorescent color dye.

In some embodiments, the environment is an in vitro environment, an in vivo environment, or an in situ environment.

Additional embodiments and features are set forth in part in the description that follows, and in part will become apparent to those skilled in the art upon examination of the specification or may be learned by the practice of the disclosed subject matter. A further understanding of the nature and advantages of the present disclosure may be realized by reference to the remaining portions of the specification and the drawings, which form part of this disclosure.

### BRIEF DESCRIPTION OF THE DRAWINGS

The description will be more fully understood with reference to the following figures, which are presented as exemplary embodiments of the invention and should not be construed as a complete recitation of the scope of the invention. It should be noted that the patent or application file contains at least one drawing executed in color. Copies of this patent or patent application publication with color drawing(s) will be provided by the Office upon request and payment of the necessary fee.

FIG. 1A illustrates double-emulsion droplets as osmotic pressure sensors in accordance with an embodiment of the invention.

FIG. 1B illustrates conceptually double emulsion droplets used as osmotic pressure sensors in cells or in the interstitial space between cells within living tissues in accordance with an embodiment of the invention.

FIGS. 2A and 2B illustrate the characterization of double-emulsion droplets at equilibrium in accordance with an embodiment of the invention.

FIGS. 2C and 2D illustrate the characterization of double-emulsion droplets at equilibrium in accordance with an embodiment of the invention.

FIGS. 2E through 2G illustrate the characterization of double-emulsion droplets at equilibrium in accordance with an embodiment of the invention.

FIG. 3 illustrates droplet volume at long timescales in accordance with an embodiment.

FIG. 4 illustrates temporal decay in fluorescence intensity in the inner droplet for different laser powers in accordance with an embodiment.

FIGS. 5A and 5B illustrate osmolality as a function of NaCl and PEG concentration in accordance with an embodiment.

FIG. 6 illustrates interfacial tension at drop interfaces in accordance with an embodiment.

FIG. 7 illustrates power law dependence of droplet equilibration time with droplet radius in accordance with an embodiment.

FIG. 8 illustrates negligible effect of FCy5 dye on relaxation kinetics in accordance with an embodiment.

FIGS. 9A through 9F illustrate pressure equilibration timescales of double-emulsion droplets in accordance with an embodiment of an invention.

FIGS. 10A through 10K illustrate in vivo and in situ measurements of osmotic pressure in blastomeres and in the interstitial fluid of zebrafish embryos in accordance with an embodiment of the invention.

FIGS. 11A through 11C illustrate fluorescence recovery after photobleaching of Dextran signal in the interstitial spaces of a developing zebrafish embryo in accordance with an embodiment.

### DETAILED DESCRIPTION

Turning to the drawings, descriptions of double emulsion droplets as osmotic pressure sensors are provided. Measuring osmotic pressure in living cells and tissues is challenging and especially difficult inside 3D multicellular systems without considerably perturbing the system. Many embodiments provide inventive methods that enable non-invasive measurements of osmotic pressure in vitro, in situ and/or in vivo. The double emulsion droplets can be used as osmotic pressure sensors in a fluidic environment. In some embodiments, the double emulsion droplets can be used for measuring osmotic pressure in cells, living cells, tissues, living tissues, organs, and organisms. The droplets can be generated using microfluidics or any other methods as appropriate to the requirements of specific applications in accordance with various embodiments of the invention. In some embodiments, the double emulsion droplets can include an aqueous core with a controlled and/or calibrated osmotic pressure, surrounded by an oil layer. This oil layer acts as a semi-permeable membrane letting water molecules go through the oil layer but not any other molecules. As a consequence, when a discrepancy in osmotic pressure between the aqueous droplet core and the surrounding environment is present, water molecules can leave or enter the core of the droplet through the oil layer. The transport of water results in a size change of the aqueous core where the size of the inner aqueous core would shrink or expand.

Many embodiments implement double emulsion droplets to measure osmotic pressure in living systems. Several embodiments calibrate the droplets by establishing the relation between applied osmotic pressure and the volume of the droplet's aqueous core. After calibration, some embodiments insert the droplets using fine glass capillaries either in cells or in between the cells of living tissues. In some embodiments, the inside of the glass capillaries can be coated with a polymer to prevent bursting the droplets. Certain embodiments monitor the size of the aqueous droplet core over time, and measure the osmotic pressure directly at the location where the droplet is located. In several embodiments, the osmotic pressure measurements can take place at the location of the droplet over a period of time. Many embodiments enable direct osmotic pressure measurement in living cells and in the extracellular spaces of living tissues (interstitial fluid). The double emulsion droplets in accordance with some embodiments can measure osmotic pressure in any biological structures where the double emulsion droplets can be injected in.

In many embodiments, double emulsion droplets can be used as non-invasive, precise and robust osmotic pressure sensors to locally measure osmotic pressure in vivo and in situ within 3D multicellular systems, such as organoids systems, synthetic multicellular systems, and/or developing embryos, both intra- and extra-cellularly. Several embodiments calibrate the double-emulsion droplets before measuring the osmotic pressure. From the calibration, some embodiments can quantify the osmotic pressure inside cells as well as in the interstitial fluid between the cells of living zebrafish embryos. The measured osmotic pressure values agree with in vitro measurements and inferences from osmotic perturbations in cell culture conditions. However, the existent methods to measure osmotic pressure require the destruction of the sample and could only obtain an average value of osmolarity for the entire tissue explant. In contrast, many embodiments implement local, in situ measurements and obtain intracellular osmotic pressure values that are constant throughout the first divisions in zebrafish embryos, and that osmotic pressures inside cells and in the extracellular spaces (interstitial fluid) are balanced.

Mechanics has been shown to affect fundamental biological processes across scales, from cellular function to organ formation and tissue homeostasis. Actomyosin force generation, cell-cell adhesion, traction forces and membrane tension have all been shown to affect cellular activity at subcellular and cellular scales. (See, e.g., D. A. Fletcher, et al., Nature, 463, 485-492, 2010; the disclosure of which is incorporated herein by reference.) At a multicellular level, active force generation and spatiotemporal control of tissue material properties have been shown to play an important role in tissue morphogenesis during embryonic development, as well as in the control of cell migration and cell differentiation. (See, e.g., Lecuit, T., et al., Annu Rev Cell Dev Biol 27, 157-184 2011; Heisenberg, C. P., et al., Cell 153, 948-962 (2013); Lenne, P. F., et al., Nat Commun 13, 664 (2022); Mongera, A., et al., Nature 561, 401-405 (2018); Barriga, E. H., et al., Nature 554, 523-527 (2018); Parada, C., et al., Dev Cell 57, 854-866.e6 (2022); Yanagida, A., et al., Cell, 185, 777-793.e20 (2022); the disclosures of which are herein incorporated by references.) Other fundamental cellular and developmental processes, such as the control of cell and nuclear sizes, cell division, cytoskeletal mechanics, the emergence of a blastocoel in early mammalian embryos, the formation of complex lumen structures during organogenesis (liver, pancreas, lung, etc.), and the emergence of gradients in extracellular spaces during embryonic development, can depend on a tight control of the osmotic pressure both inside cells and in the extracellular space. (See, e.g., Lemiere, J., et al., Elife 11, e76075 (2022); Mitchison, T. J., Mol Biol Cell 30, 173-180 (2019); Stewart, M. P. et al., Nature 469, 226-230 (2011); Guo, M. et al., Proc Natl Acad Sci USA 114, E8618-E8627 (2017); Dumortier, J. G. et al., Science 365, 465-468 (2019); Mosaliganti, K. R. et al., Elife 8, e39596 (2019); Navis, A., et al., Semin Cell Dev Biol 55, 139-147 (2016); Bagnat, M., et al., Annu Rev Cell Dev Bi 38, 375-394 (2022); Schliffka, M. F., et al., Curr Opin Genet Dev 57, 70-77 (2019); Chugh, M., et al., Semin Cell Dev Biol 131, 134-145 (2022); Li, Y., et al., J Cell Sci 133, jcs240341 (2020); the disclosures of which are herein incorporated by references). Yet, measuring osmotic pressure remains very challenging, especially in 3D multicellular systems such as living tissues or organoids, hindering the understanding of the role that osmotic pressure plays in living organisms.

Previous measurements of intracellular hydrostatic pressure in cells in culture conditions (in vitro) or in externally accessible lumens in vivo has been achieved using either microneedles as a pressure gauge or other surface contact probes, such as Atomic Force Microscopy. (See, e.g., Jones, T. M., et al., Phys Biol 18, 066003 (2021); Chan, C., et al., Development 147, dev181297 (2020); Lamire, L.-A. et al. Plos Biol 18, e3000940 (2020); the disclosures of which are herein incorporated by references.) These techniques require an external probe to be in constant contact with the sample, which is invasive and not well-suited for 3D multicellular systems that continuously change shape. Previous microdroplet-based techniques have been developed to measure mechanical stresses or material properties in situ and in vivo, but these do not allow measurements of (osmotic) pressure. (See, e.g., Campas, O. et al. Nat Methods 11, 183-189 (2014); Serwane, F. et al. Nat Methods 14, 181-186 (2017); the disclosures of which are herein incorporated by references.) Gel beads can perform measurements of isotropic stress associated with cellular crowding in multicellular systems, but cannot measure osmotic pressure either. (See, e.g., Traber, N. et al. Sci Rep 9, 17031 (2019); Mohagheghian, E. et al. Nature Communications 9, 1-14 (2018); the disclosures of which are incorporated herein by references.) Finally, measurements of the interstitial fluid osmolarity in early zebrafish embryos were achieved using standard osmometers by collecting large interstitial fluid quantities in whole tissue explants. (See, e.g., Krens, S. F. G. et al. Development 144, 1798-1806 (2017); the disclosure of which is herein incorporated by reference.) These measurements provided an average value of interstitial fluid osmolarity for the entire explant, which required the destruction of the sample, thereby precluding any measurements of spatial or temporal variations in osmotic pressure in the tissue. Measuring osmotic pressure locally in situ and in vivo, within cells or tissues of developing embryos (including lumen formation in organogenesis) or in other 3D multicellular systems such as organoids, remains challenging.

Many embodiments provide inventive realization of osmotic pressure sensors. In some embodiments, the sensors can measure the osmotic pressure in vitro, in vivo, and/or in situ. In several embodiments, the osmotic pressure sensors are able to quantify osmotic pressure in cells as well as in the interstitial fluid between cells of 3D living tissues. In some embodiments, the osmotic pressure sensors can measure and monitor osmotic pressures in any 3D multicellular systems including (but not limited to): living tissues, developing embryos, tissue explants, multicellular spheroids, lumen in embryos or in cell aggregates in vitro, cell aggregates, organoids, gastruloids, and all synthetics multicellular systems (such as truncoids).

In several embodiments, the osmotic pressure sensors comprise double emulsion microdroplets. The double emulsion microdroplets in accordance with some embodiments can be made of a biocompatible oil droplet containing at least one smaller aqueous droplet with a calibrated concentration of osmolyte. In certain embodiments, the oil droplet contains multiple aqueous droplets inside. In many embodiments, the inner aqueous droplets can include molecules and/or osmolytes that have molecular weight that are greater than about 6,000 Dalton. In various embodiments, the osmolytes for inner aqueous droplets are water soluble. Examples of osmolyte for inner aqueous droplets include (but are not limited to): polyethylene glycol (PEG), Dextran, any water-soluble molecules with desired molecular weight, and ions. In several embodiments, the types of osmolytes can be selected such that the double emulsion droplets can be used to measure different types of osmotic pressure associated with different osmolytes. In certain embodiments, the oil surrounding the inner aqueous droplet can act as a protective shell while allowing water transport, effectively behaving as a water permeable layer. The oil shell can be modified with various surfactants to form micelles and facilitate water transport. The oil shell can be made of biocompatible oils including (but not limited to) fluorocarbon oils, silicone oils, or biocompatible oils. In several embodiments, the surfactants can facilitate water transport through the oil shell. In a number of embodiments, the surfactants can be selective to desired types of molecules such that the double emulsion droplets can be used to measure different types of osmotic pressure associated with different osmolytes. Examples of surfactants include (but are not limited to): molecular components with hydrophilic parts and hydrophobic parts, lipids, sodium dodecyl sulfate, sodium lauryl sulfate, poly(N-isopropylacrylamide), and fluorinated surfactants. Some embodiments use fluorinated surfactants comprising perfluoropolyether (PFPE), polytetrafluoroethylene (PTFE), fluorine, carbon and oxygen. Examples of fluorinated surfactants include (but are not limited to) Krytox™-PEG (600), Krytox™, and perfluoro-15-crown-5-ether. As can be readily appreciated, any of a variety of surfactants can be utilized as appropriate to the requirements of specific applications in accordance with various embodiments of the invention.

In many embodiments, the outer droplet of the double emulsion droplets can have an average diameter of: less than about 5 microns; or less than about 10 microns; or less than about 15 microns; or less than about 20 microns; or less than about 30 microns; or less than about 40 microns; or less than about 50 microns; or less than about 60 microns; or less than about 70 microns; or less than about 80 microns; or less than about 100 microns; or less than about 120 microns; or less than about 140 microns; or less than about 160 microns; or greater than about 1 microns; or greater than about 5 microns; or greater than about 10 microns; or greater than about 20 microns; or greater than about 30 microns; or greater than about 40 microns; or greater than about 50 microns. As can be readily appreciated, any of a variety of droplet diameter can be utilized as appropriate to the requirements of specific applications in accordance with various embodiments of the invention. The inner droplets of the double emulsion droplets can have an average diameter of less than the outer droplet diameter.

Several embodiments control various parameters including (but not limited to) the osmolyte concentration in the inner droplet, the surfactants on the oil shell, and the relative inner/outer droplet sizes, to generate osmotic pressure sensors with well-defined characteristics. Many embodiments monitor the size change of the double emulsion droplets as a reflection of the osmotic pressure. Several embodiments measure the droplet size change using microscopes. Examples of microscopes include (but are not limited to): optical microscope, fluorescence microscope, confocal microscope, super resolution microscope, light-sheet microscope, and any microscopy technique that allow the visualization of the droplet. As can be readily appreciated, any of a variety of microscope can be utilized as appropriate to the requirements of specific applications in accordance with various embodiments of the invention. In a number of embodiments, water soluble colored dyes and/or fluorescent dyes can be added to the inner aqueous droplets to assist the monitoring process. In certain embodiments, the dyes can be added to both the oil shell and the inner aqueous droplets for imaging. After calibrating the sensors, a number of embodiments use the sensors to measure the osmotic pressure in blastomeres (cells) of early zebrafish embryos, and/or in the interstitial fluid between the cells of the zebrafish blastula by monitoring the size of droplets previously inserted in the embryo. The osmotic pressure measurement results show a balance between intracellular and interstitial osmotic pressures, with values of approximately 0.7 MPa, but a large pressure imbalance between the inside and outside of the embryo. The osmotic pressures in the embryo are about 17-fold higher than that of the medium external to the embryo, showing that embryos are able to maintain a large osmotic pressure difference between their interior and the surrounding environment. These results suggest that osmolarity is highly regulated both in cells and in the interstitial fluid, in agreement with the observations showing a much smaller variability in osmotic pressure within a given embryo than across different embryos. Several embodiments employ droplets with high interfacial tensions that do not allow cells in zebrafish embryos to deform them. Some embodiments may use double emulsion droplets to measure simultaneously the local osmotic pressure and the anisotropic mechanical stresses in the tissue by simultaneously monitoring the volume of the inner droplet and the shape deformations of the outer droplet, respectively.

In many embodiments, double-emulsion droplets enable in vitro, in situ and in vivo measurements of osmotic pressure, both intra- and extra-cellularly within cells, living cells, tissues, and living tissues. The ability to measure osmotic pressure in 3D multicellular systems (developing embryos, organoids, such as pancreas, liver, and other organ systems.) will help understand its role in fundamental biological processes.

Osmotic pressure can play an import role in tissue homeostasis (from skin to the vascular system) and also in cancer progression. There is medical interest in measuring osmotic pressure in a wide range of tissues. Applications in the beauty industry are likely, since skincare relies on the water content of the tissue (skin hydration), which can be directly related to osmolarity. In many embodiments, the double emulsion droplets can be used to measure the osmotic pressure in reconstituted skin directly and test how skin care products affect the osmotic pressure in the tissue. The double emulsion droplets could be used to quantify the osmotic pressure in cosmetic products, as cosmetic products are in the form of foams, emulsions, gels or colloidal suspensions and their stability can depend on the osmotic pressure. In the medical industry, the double emulsion droplets in accordance with several embodiments can be used to measure osmolarity in vasculature (both in healthy conditions and in the wide range of diseases associated with changes in the osmotic pressure of the vascular system, such as hemodynamic disorders) and in other organs such as kidneys, to the evolution of tumors, where osmotic pressures changes may occur. In various embodiments, the double emulsion droplets can be used as delivery agents for drugs and/or other chemicals that could be locally released in tissues. A number of embodiments can modify the type of surfactants, the concentration of surfactants, and/or the droplets sizes, to create osmotic pressure sensors with different capabilities. For food industry, the double emulsion droplets in accordance with many embodiments can measure of osmotic pressure and/or related quantities, such as the ionic strength, in soft foods. The industrial preparation of different foods, such as ice cream and sauces, relies on the stability of emulsions and foams which are influenced by the osmotic pressure. These properties can also be important in the generation of artificial meats (lab grown or meat alternatives). The ability to measure osmotic pressure in these systems could help formulation development to improve texture of food, test and monitor food quality in artificial or processed foods. The double emulsion droplets enable direct measurements of the texture within foods of small quantities.

### Double Emulsion Microdroplets as Osmotic Pressure Sensors

Many embodiments implement double emulsion droplets, including an aqueous droplet embedded in an oil shell (water-in-oil-in-water, or W/O/W double emulsions) as osmometers since water flux through the oil shell is possible in the presence of surfactants. The water transport through the fluorocarbon oil layer can rely on inverse micelles formed by the surfactant within the oil layer. The outer water-permeable oil layer enables the inner aqueous droplet to increase or decrease its volume as water enters or leaves the droplet, respectively. Changes in osmolarity in the external medium can drive water flows through the oil shell of the double-emulsion droplet, indicating that the system is sensitive to osmotic pressure differences. In order for double-emulsion droplets to be used as osmotic pressure sensors, several embodiments control the osmolarity and size of the inner aqueous droplet, as well as the size and surfactant composition of the outer oil layer to enable the generation of stable and calibrated double emulsion droplets.

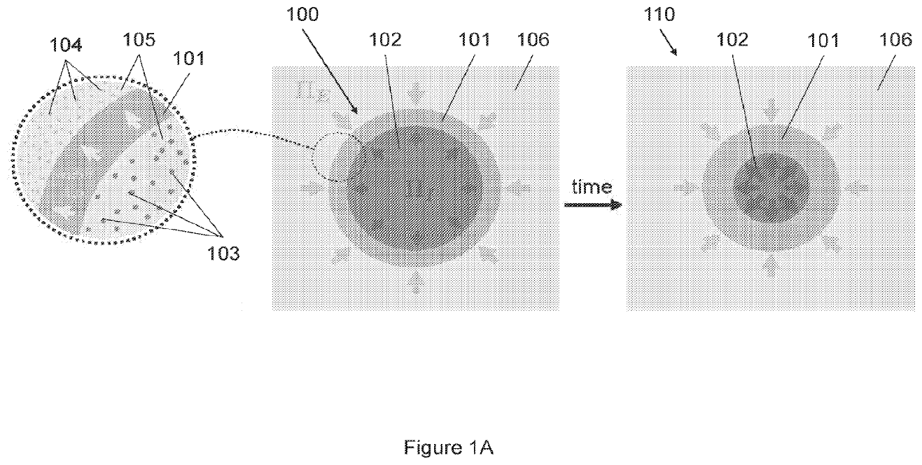

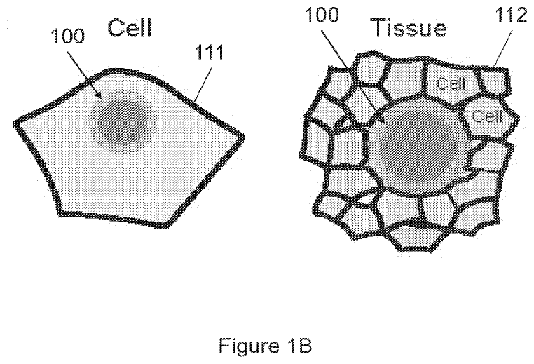

FIG. 1A illustrates double-emulsion droplets as osmotic pressure sensors in accordance with an embodiment of the invention. The double emulsion droplets 100 can include an oil shell 101 and an aqueous droplet 102 inside the oil shell. The inner aqueous droplet 102 can contain an osmolyte 103. The osmolyte 103 in the inner aqueous droplet 102 can have a controlled and/or calibrated composition and/or concentration. The oil shell 101 can be modified with various surfactants (not shown) to form micelles and facilitate water transport. The double emulsion droplets 100 can be placed in an external medium 106. The external medium can include osmolytes 104 such as (but not limited to) ions, metabolites, etc. dissolved in an aqueous environment. If the osmolyte 104 concentration of the external medium 106 is higher than the inner osmolyte 103, water 105 can transport through the oil shell 101 from the inner aqueous droplet 102 to the external medium 106 to reach an equilibrium, and the double emulsion droplets 100 would shrink in size as shown in 110. If the osmolyte concentration of the external medium 104 is lower than the inner osmolyte 103, water 105 can transport through the oil shell 101 from the external medium 106 to the inner aqueous droplet 102 to reach an equilibrium, and the double emulsion droplets would expand in size (not shown). The inner osmolyte 103 can be controlled and calibrated to monitor the size change of the droplet and the osmotic pressure.

FIG. 1B illustrates conceptually double emulsion droplets used as osmotic pressure sensors in cells or in the interstitial space between cells within living tissues in accordance with an embodiment of the invention. FIG. 1B illustrates a double emulsion droplet 100 inside a cell 111, and a double emulsion droplet 100 inside in the extracellular space between cells 112. The double emulsion droplets 100 enable measurements of the intracellular osmotic pressure and of the osmotic pressure of the extracellular interstitial fluid, respectively.

Several embodiments use microfluidics to produce monodispersed, stable water-in-oil-in-water double-emulsion droplets. Microfluidics enable control over the initial volumes of both the inner aqueous droplet and outer oil layer within the range (such as about 10-40 μm in outer droplet radius). In some embodiments, biocompatible fluorocarbon oils can be used for the oil phase and non-ionic fluorinated surfactants (such as Krytox-PEG) can be used to stabilize the droplets. In certain embodiments, fluorinated fluorophore can be used to visualize the oil layer using fluorescence microscopy. In order to control the osmotic pressure of the inner aqueous droplet, several embodiments use PEG (a small fraction of it being fluorescently-labeled) as a water soluble non-ionic osmolyte during the generation of droplets. Microfluidic generation of such droplets in a polyvinyl alcohol (PVA) solution of fixed osmolarity can lead to stable double emulsions with controlled initial volumes. The fluorescent dyes in the inner droplet and the oil layer permit the quantification of inner/outer droplet sizes at high resolution using fluorescence microscopy in accordance with several embodiments.

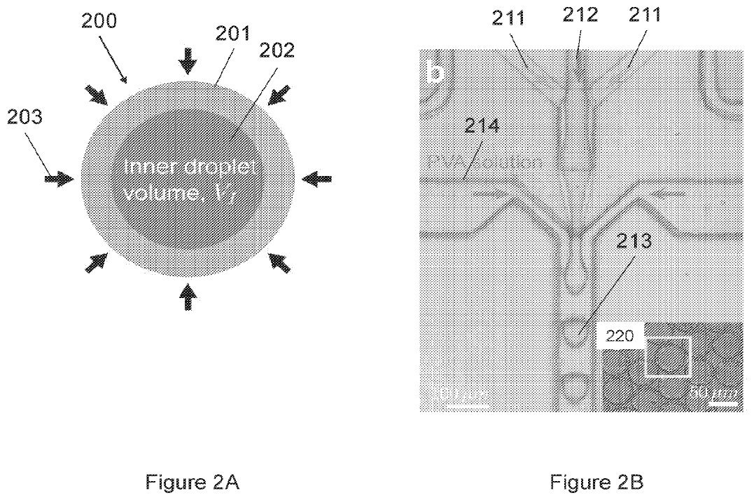

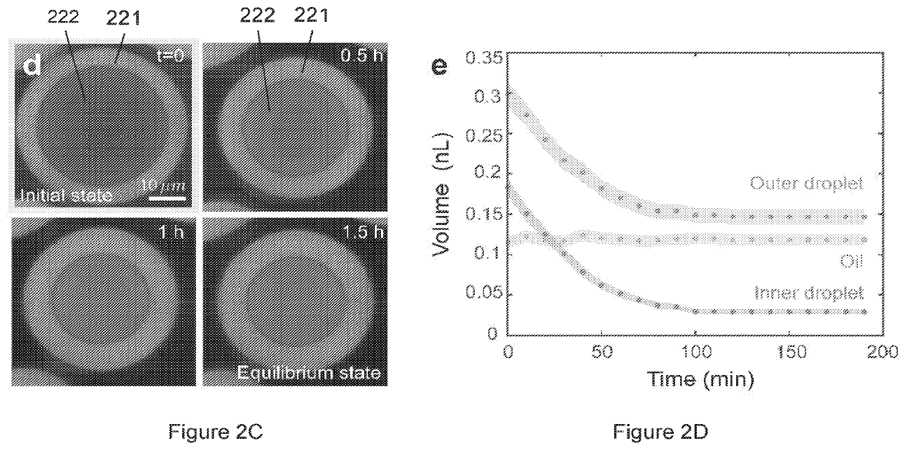

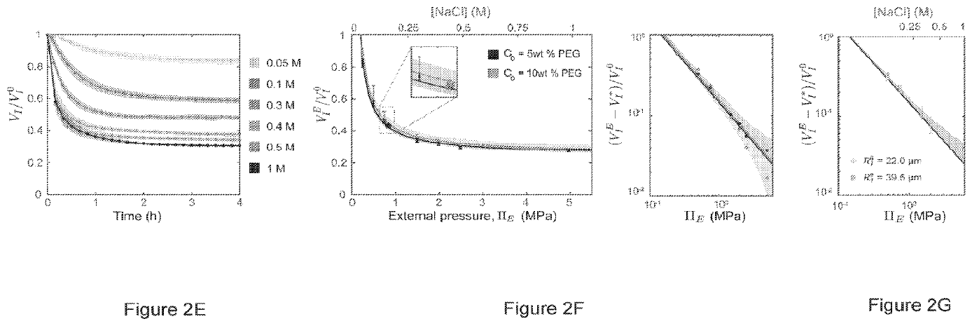

FIGS. 2A and 2B illustrate the characterization of double-emulsion droplets at equilibrium in accordance with an embodiment of the invention. FIG. 2A conceptually illustrates a double emulsion droplet indicating its composition and characteristics. The double emulsion droplet 200 comprises a biocompatible fluorocarbon oil layer 201. The fluorocarbon oil shell 201 can be functionalized with surfactants such as (but not limited to) Krytox-PEG (not shown). A water droplet 202 (or an aqueous droplet) with water soluble osmolyte locates inside the oil layer 201. The inner water droplet 202 can have a volume of V_{I}. The inner water droplet can be labelled with fluorescent dye such as (but not limited to) fluorescent PEG to assist visualization. The double emulsion droplet 200 is susceptible to external osmotic pressure Π_{E}, 203.

FIG. 2B illustrates microfluidic generation of double-emulsion droplets. The biocompatible fluorocarbon oil 211 (with non-ionic fluorinated surfactants (such as Krytox-PEG)) can be mixed with aqueous solution 212 (with water soluble osmolyte) via a plurality of inlets of the microfluidic platform to produce monodispersed, stable W/O/W double-emulsion droplets 213. Microfluidic generation of such droplets 213 in a PVA solution 214 of fixed osmolarity can lead to stable double emulsions with controlled initial volumes. 220 shows generated double emulsion droplets with scale bar of about 50 μm.

FIGS. 2C and 2D illustrate the characterization of double-emulsion droplets at equilibrium in accordance with an embodiment of the invention. FIG. 2C shows a confocal section of a double-emulsion droplet showing the temporal reduction in inner and outer droplet sizes when placed in a 0.4 M NaCl solution. Fluorocarbon oil 221 and fluorescent PEG 222 are shown. Scale bar is about 10 μm. FIG. 2D illustrates temporal evolution of the inner droplet volume, V_{I}, the outer droplet volume, V_{T} and the oil layer volume. Error bands are measurement error for single droplet.

Many embodiments characterize the response of double-emulsion droplets to controlled changes in osmolarity in the external medium. Several embodiments place double-emulsion droplets in an aqueous medium containing a salt (such as NaCl) concentration of about 0.4 M can drive a progressive and strong reduction in droplet volume as water left the inner droplet through the oil layer (FIG. 2C). This can lead to an increase in the fluorescent intensity signal in the inner droplet as fluorescent PEG becomes more concentrated. Monitoring the reduction in the volumes of both inner and outer droplets show that both decrease equally over time from their respective initial volumes, eventually reaching equilibrium volumes as the pressure in the inner droplet equilibrates with the external pressure (FIG. 2D). Throughout this process, the oil shell volume remains constant (FIG. 2D), indicating that monitoring the inner or outer droplet volume provides the same information about droplet sizes. The fact that the reduction in volume can reach an equilibrium value indicates that only water is transported through the oil shell. Long term (12 h) imaging of double-emulsion droplets at varying laser intensities displayed a laser power dependent decay in PEG signal intensity, indicating that the slight observed decay is mostly due to photobleaching rather than PEG leakage from the inner droplet, in agreement with the constant equilibrium droplet volume at long timescales. These results provide that double-emulsion droplets can be used as proper osmotic pressure sensors in accordance with many embodiments.

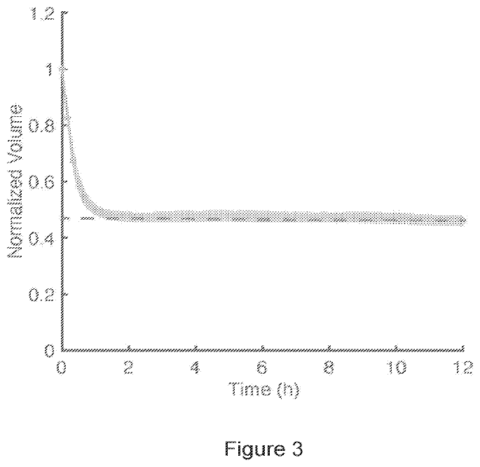

FIG. 3 illustrates droplet volume at long timescales in accordance with an embodiment. Inner droplet volume normalized to its initial volume as a function of time (initial radius, 38 μm). Double emulsion droplets are placed in cell culture media and monitored for 12 h (N=13). After its initial relaxation to its equilibrium value, the equilibrium droplet volume does not show any significant change over these long time periods. Mean±SD (standard deviation, shown as error band).

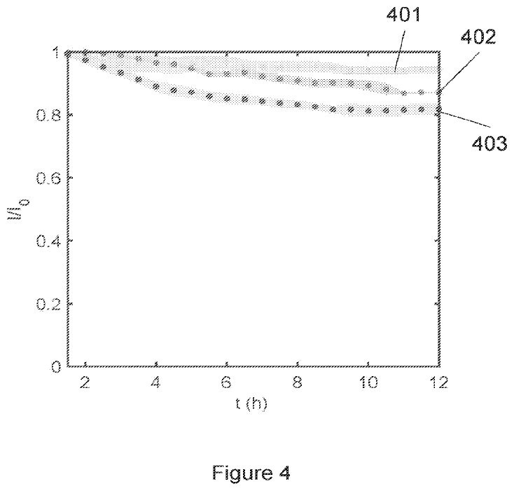

FIG. 4 illustrates temporal decay in fluorescence intensity in the inner droplet for different laser powers in accordance with an embodiment. Temporal decay of the emission intensity I normalized to the initial intentity I₀ for droplets in a 0.4M NaCl solution, for different laser powers: 0.002 mW (401; N=6), 0.004 mW (402; N=4), and 0.006 mW (403; N=5). All other imaging parameters are the same. After 12 h, the inner core intensity is 97% of the maximum intensity at 0.002 mW laser power intensity. The reduction in fluorescence intensity after 12 h increases with the laser power of excitation illumination, suggesting that the reduction in intensity is caused by photobleaching rather than PEG exiting the inner droplet. Even if there is leakage of PEG, these results indicate that PEG loss would be limited to 3% over 12 h. Mean±SD (shown as error band).

FIGS. 2E through 2G illustrate the characterization of double-emulsion droplets at equilibrium in accordance with an embodiment of the invention. FIG. 2E illustrates temporal evolution of the inner droplet volume, V_{I} (normalized by the initial volume

$${V}_{I}^{0}$$

of the inner droplet at equilibrium with a 10% w/w PVA solution;

$${R}_{I}^{0} = 33.5 \pm 0.6 µm , with {V}_{I}^{0} = ( 4 π / 3 ) {( {R}_{I}^{0} )}^{3} ) ,$$

for double-emulsion droplets with fixed initial internal PEG concentration placed in salt solutions of varying osmolarities. Error bands are SD. N=20 (0.05 M), 16 (0.1 M), 16 (0.3 M), 15 (0.4 M), 21 (0.5 M), 17 (1 M). FIG. 2F shows measured dependence of the equilibrium inner droplet volume,

$${V}_{I}^{E} ( normalized by {V}_{I}^{0} ) ,$$

on the externally imposed osmolarity (or osmotic pressure, Π_{E}) for droplets with 5% w/w (black circles) and 10% w/w (red circles) initial PEG concentrations. The top inset shows the power-law dependence of the normalized osmotically-active equilibrium volume of the inner droplet,

$$( {V}_{I} - {V}_{I}^{*} ) / {V}_{I}^{0} ,$$

on the external osmotic pressure, Π_{E}, for both PEG initial concentrations. Lines are fits of Eq. 1 to the data for each initial PEG concentration. The small inset shows a magnified region of FIG. 2F with the measured equilibrium volumes of the inner droplet of double-emulsion droplets with 5% w/w (black star) and 10% w/w (red star) initial PEG concentrations when placed in cell culture media of known osmolarity. N=13 (5 wt %), 25 (10 wt %). Fit CBs (68%) are shown. Error bars are SD. FIG. 2G illustrates measured dependence of the equilibrium inner droplet volume,

$${V}_{I}^{E} ,$$

on the externally imposed osmolarity (or osmotic pressure, Π_{E}) for droplets of different initial sizes

$$( large droplets : {R}_{1}^{0} = 33.5 \pm 0.6 µm , \\ blue ; small droplets : {R}_{I}^{0} = 12.2 \pm 0.3 µm , green )$$

but same initial PEG concentration (5% w/w). Black line is the calibration curve (fit in FIG. 2G) for 5% w/w PEG. Small droplets: N=26 (0.5 MPa), 23 (0.75 MPa), 18 (1.5 MPa), 23 (2 MPa). Large droplets: 16 (0.5 MPa), 16 (0.75 MPa), 15 (1.5 MPa), 22 (2 MPa). CB (68%) of calibration curve is shown. Error bars are SD.

To test the sensitivity of double-emulsion droplets to different external osmotic pressures, several embodiments monitor the temporal evolution of their inner droplet volume V_{I} when placed in solutions with different salt (such as NaCl) concentrations in the range from about 0.05 M to about 1 M (FIG. 2E). The osmolality of each of these solutions can be measured using a commercial osmometer to obtain the osmotic pressure Π_{E} of each solution (ranging from 0.25 to 4.96 MPa). These salt solutions of known osmolalities (and osmotic pressures) can be used to calibrate the double emulsion droplets. For all concentrations, the inner droplet volume decreased over time from its initial volume

$${V}_{I}^{0}$$

until reaching an equilibrium volume

$${V}_{I}^{E}$$

that depend on the externally imposed osmotic pressure Π_{E}, with larger osmotic pressures leading to smaller equilibrium volumes (FIG. 2E). Measurements of the inner and outer droplet interfacial tensions allow an estimation of the droplet capillary stresses (both approximately of 1 kPa), which are several orders of magnitude smaller than the measured osmotic pressures and, consequently, do not affect the measurements. The equilibrium volume of the inner droplet showed a power law dependence on the external pressure (FIG. 2F and top inset), albeit not becoming smaller than a minimal volume

$${V}_{I}^{*} ,$$

associated with PEG volume exclusion (osmotically inactive volume). This power law relation is consistent with the inner droplet's osmotic pressure

$${\prod }_{I} = A / ( {V}_{I} - {V}_{I}^{*} )$$

being equal to the external osmotic pressure at equilibrium, namely

$${Π}_{E} = A / ( {V}_{I}^{E} - {V}_{I}^{*} ) \qquad Eq . 1$$

where A is a constant associated with the inner droplet osmolyte concentration and can be related to the initial conditions of droplet preparation by

$$A = {Π}_{I}^{0} / ( {V}_{I}^{0} - {V}_{I}^{*} ) , with {Π}_{I}^{0}$$

being the osmotic pressure of the initial PVA solution, fixed at 79 mOsm/kg or 0.2 MPa. Double-emulsion droplets with different initial PEG concentrations in the inner droplet also follow Eq. 1 (FIG. 2F). To test if this same relation holds in the presence of more complex external chemical environments, some embodiments place double-emulsion droplets in cell culture media. The resulting equilibrium inner droplet volumes follow the same relation in cell culture media as for simple salt solutions with the same osmotic pressure, regardless of the initial PEG concentrations in the inner droplet (FIG. 2F, small inset). The same behavior can be observed for different initial inner droplet volumes at fixed PEG concentration in the inner droplet (FIG. 2G). These results indicate that the power law relation in Eq. 1 constitutes a robust calibration curve of double-emulsion droplets, providing the relation between the measured inner droplet volume and the osmotic pressure in the external medium at equilibrium.

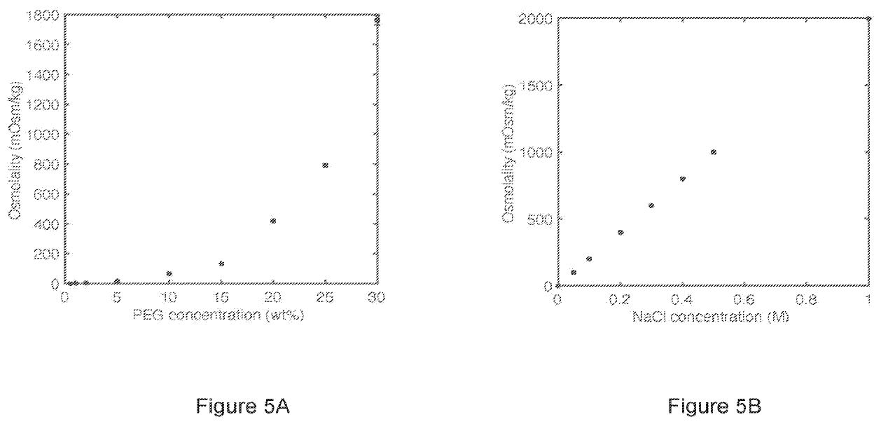

FIGS. 5A and 5B illustrate osmolality as a function of NaCl and PEG concentration in accordance with an embodiment. Measurements of osmolality for (FIG. 5A) PEG 6000 and (FIG. 5B) salt (NaCl) solution for varying PEG and NaCl concentrations respectively. N=3 for each concentration value. Mean±SD (error bars are too small to see).

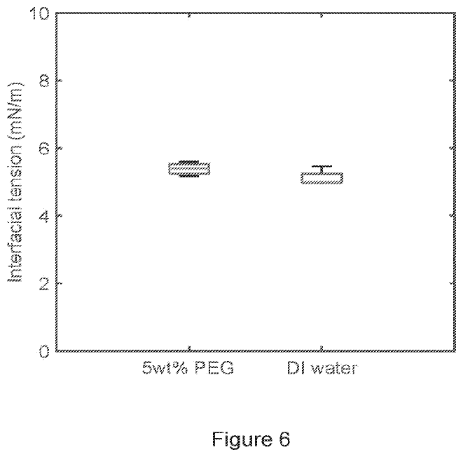

FIG. 6 illustrates interfacial tension at drop interfaces in accordance with an embodiment. Interfacial tension values for HFE7700 with 2% w/w KP600 in DI water (outer droplet interface) and in an aqueous solution of PEG6000 (5% w/w) in DI water (inner droplet interface). N=4 and 5, respectively. Boxplot show Median, 25th and 75th percentiles, whiskers extend to extreme data points.

Beyond equilibrium values, to evaluate the temporal resolution of the measurements, it is important to know the relaxation timescale τ_{R} of pressure equilibration in double-emulsion droplets. Some embodiments monitor the volume of the inner droplet over time and measure the dependence of the relaxation timescale on the different control parameters, such as the initial inner droplet radius,

$${R}_{I}^{0} ,$$

initial internal pressure,

$${Π}_{I}^{0} ,$$

imposed external pressure, Π_{E}, and the initial oil volume fraction

$${V}_{oil} / {V}_{T}^{0}$$

(with Vₒᵢₗ being the volume of oil on the membrane and

$${V}_{T}^{0}$$

the Initial volume of the entre droplet). The relaxation timescale τ_{R} displayed a strong dependence on the initial inner droplet size

$${R}_{I}^{0} ,$$

with increasing relaxation time for increasing droplet sizes. This behavior is compatible with a power law dependence of the relaxation timescale τ_{R} on

$${R}_{I}^{0} .$$

While smaller values of the initial inner pressure

$${Π}_{I}^{0}$$

can lead to shorter relaxation timescales, pressure equilibration occurred faster for larger external pressures Π_{E}. Finally, no Call NO dependence of the relaxation timescale on the oil volume fraction

$${V}_{o i l} / {V}_{T}^{0}$$

can be observed, likely because there is always a region where the oil layer thickness is small due to the inner droplet buoyancy. The measured values of τ_{R} were not affected by the presence of fluorinated dye in the fluorocarbon oil. In order to perform measurements of osmotic pressure on relatively short timescales (˜10 min), the initial radius of the inner droplet

$${R}_{I}^{0}$$

should pe smaller than approximately 20 μm and have small initial internal pressures (less than about 100 kPa). In many embodiments, droplets with characteristics (such as

$${R}_{I}^{0}$$

less than about 20 μm and

$${\prod }_{I}^{0}$$

less than about 100 kPa for osmotic pressure measurements in living embryos.

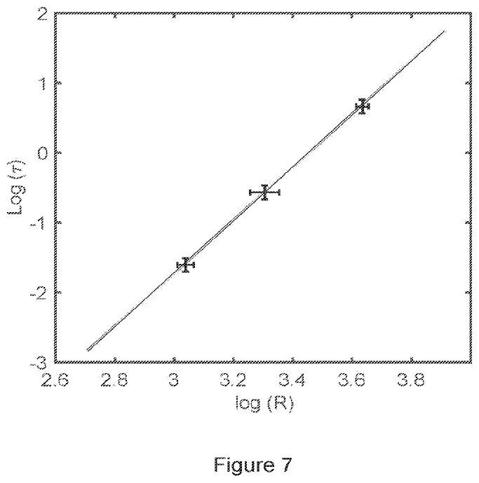

FIG. 7 illustrates power law dependence of droplet equilibration time with droplet radius in accordance with an embodiment. Log-log plot of the characteristic time, τ, as a function of the initial radius of the inner droplet, R, showing that the data follows a power law dependence (linear in log-log scale). Linear fit is used to obtain the exponent of the power law (y=ax+b, with a=3.8±0.5 and b=−13.1±2). Mean±SD (both in x and y). N=20, 20, 47 droplets (from left to right).

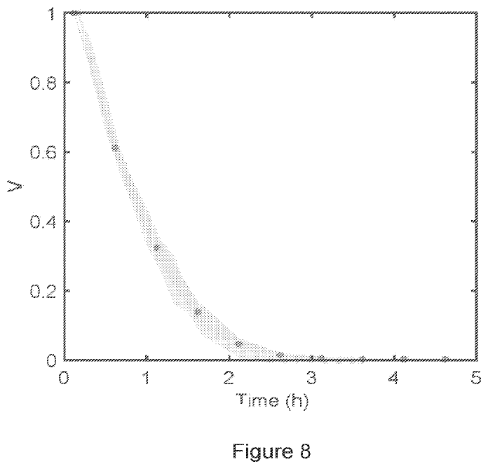

FIG. 8 illustrates negligible effect of FCy5 dye on relaxation kinetics in accordance with an embodiment. Temporal evolution of the normalized inner droplet volume V in a 0.4M NaCl solution both in the presence (0.025 mM; green; N=5) and absence (red; N=3) of FCy5 dye in the fluorocarbon oil phase. Initial average droplet radius is 43 μm in both cases. Mean±SD (shown as error band).

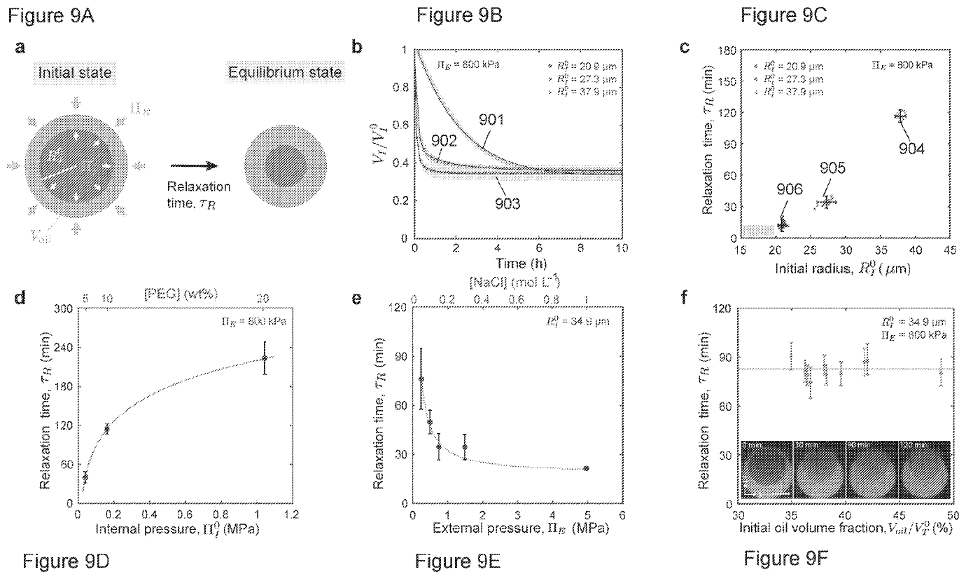

FIGS. 9A through 9F illustrate pressure equilibration timescales of double-emulsion droplets in accordance with an embodiment of an invention. FIG. 9A illustrates a double-emulsion droplet of initial inner pressure

$${\prod }_{I}^{0}$$

and volume

$${V}_{I}^{0} ( or radius {R}_{I}^{0} )$$

and initial oil volume

$${V}_{oil}^{0} ,$$

reducing its volume to the equilibrium values over a timescale τ_{R}. FIG. 9B illustrates inner droplet volume relaxation

$$( normalized to the initial volume {V}_{t}^{0} )$$

for double-emulsion droplets of different initial sizes:

$${R}_{I}^{0} = 37. 9 \pm 0.7 μm , \qquad 901 ;$$

$${R}_{I}^{0} = 27.3 \pm 0.4 μm , \qquad 902 ;$$

$${R}_{I}^{0} = 20. 9 \pm 0.3 μm , \qquad 903$$

(initial PEG concentration (5% w/w) and fixed Π_{E}). Lines are exponential fits to the data. FIGS. 9C through 9F show dependence of the measured equilibrium relaxation timescale on the initial inner droplet size,

$${R}_{I}^{0}$$

(FIG. 9C; initial PEG concentration (5% w/w) and fixed Π_{E}; N=47 (904),20 (905), 20 (906)), the initial internal pressure,

$${\prod }_{I}^{0} .$$

FIG. 9D: fixed

$${R}_{I}^{0}$$

and Π_{E}, N=13 (5 w/w), 18 (10 w/w %), and 20 (20% w/w)), the externally imposed osmolarity, Π_{E}. FIG. 9E: initial PEG concentration (5% w/w) and fixed

$${R}_{I}^{0} ,$$

N=11 (0.25 MPa), 15 (0.5 MPa), 12 (0.75 MPa), 14 (1.5 MPa) and 16 (5 MPa)), and the initial oil volume fraction.

$${V}_{oil}^{0} / {V}_{T}^{0} .$$

FIG. 9F: initial PEG concentration (10% w/w), fixed

$${R}_{I}^{0} ,$$

and Π_{E}), with

$${V}_{T}^{0}$$

being the initial total droplet volume. Inset in FIG. 9F shows z-x confocal sections of a droplet relaxing to the equilibrium state; Scale bar, 25 μm. All error bars are SD.

Since double emulsion osmotic pressure sensors need to equilibrate their volume to read the local osmotic pressure, the time resolution of the measurements may be limited. For measurements in vivo and in situ with droplets of approximately 30 μm in diameter, the equilibration timescale is less than 10 min. Faster equilibration times are possible for smaller droplets, enabling faster measurements. The timescale of equilibration can also be affected by the ability of fluid to move in the tissue. In the measurements, fluid is able to move between cells at timescales shorter than droplet volume equilibration. However, the equilibration timescale of the droplet could potentially become limited by fluid availability in its neighborhood in tissues with very small interstitial spaces.

### In Situ and In Vivo Measurements of Osmotic Pressure in Zebrafish Blastomeres

Many embodiments use double emulsion droplets to measure the osmotic pressure inside blastomeres (cells) of developing zebrafish embryos. A single double-emulsion droplet can be injected in the embryo at the 1-cell stage using a fine glass capillary. To measure the local value of the osmotic pressure, several embodiments monitor the volume of the inner droplet over time for over 3 hours, from the 4-cell stage until the cell size becomes approximately twice the droplet size. The measured intracellular osmotic pressure values are of about 280 mOsm/kg (0.7 MPa) on average and remain largely constant throughout the measurement period. The measured intracellular osmotic pressure should change to the osmotic pressure of the external medium (E3 buffer; Methods) upon dissolution of cell membranes, since the double emulsion droplet would progressively be exposed to the external embryo E3 medium. Some embodiments use about 2% (w/w) sodium dodecyl sulfate (SDS) to dissolve cells' membranes and completely disperse their contents in the external medium. The osmotic pressure is monitored during the process and found to progressively decrease from its measured intracellular value to the osmotic pressure of the external embryo E3 medium in the presence of 2% w/w SDS, which is approximately 5-fold smaller than the intracellular osmotic pressure. These results indicate that double emulsion droplets accurately measure the local osmotic pressure, and that cells (blastomeres) in early embryos tightly regulate their intracellular osmotic pressure through division cycles (cleavages).

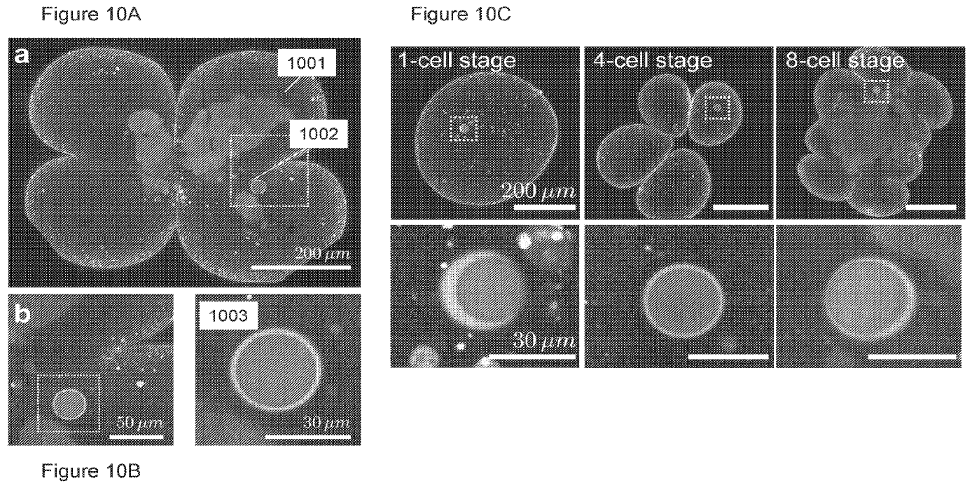

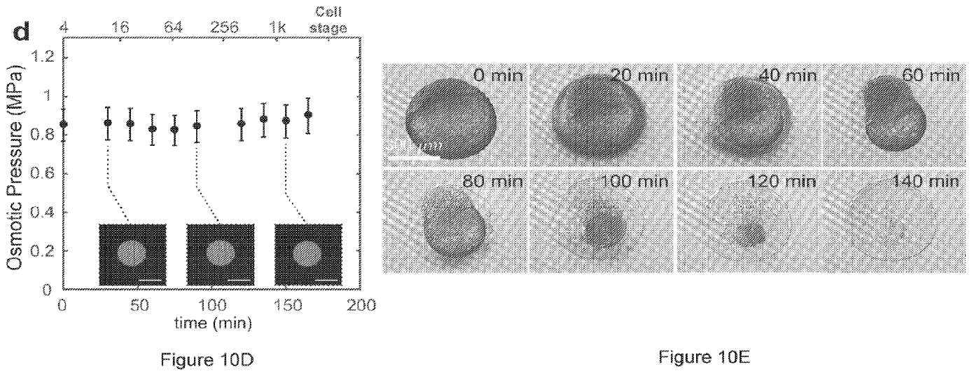

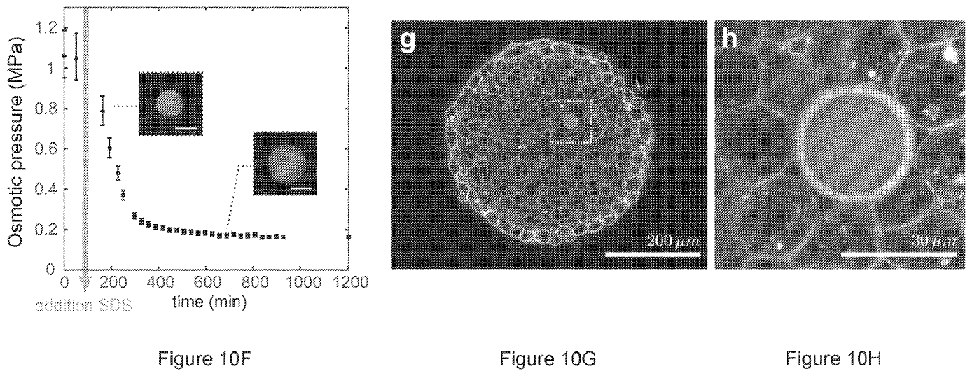

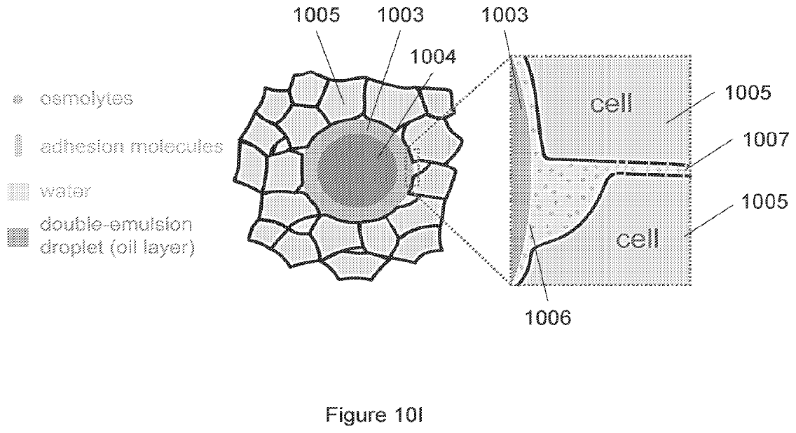

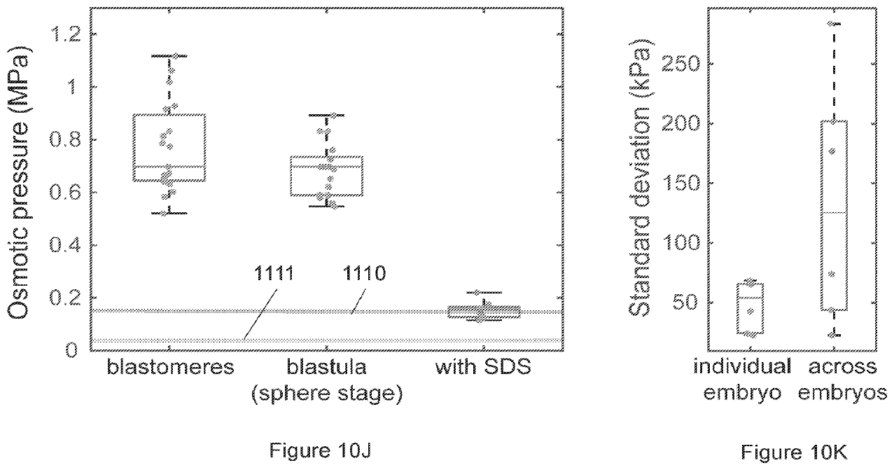

FIGS. 10A through 10K illustrate in vivo and in situ measurements of osmotic pressure in blastomeres and in the interstitial fluid of zebrafish embryos in accordance with an embodiment of the invention. FIG. 10A illustrates confocal section of zebrafish embryo transitioning from the 2- to 4-cell stages (membranes, 1001) with a double-emulsion droplet 1002 (fluorescent PEG in inner droplet with a fluorocarbon oil shell) located in one of the blastomeres (cells). FIG. 10B show close ups of the double emulsion droplet in FIG. 10A. 1003 shows an enlarged image of the double emulsion droplet in FIG. 10B. FIG. 10C shows confocal images of a double-emulsion droplet inside a cell of a developing zebrafish embryo at different developmental stages. Scale bars, 200 μm. Close ups of the double emulsion droplet at each stage. Scale bars, 20 μm. FIG. 10D shows measured time evolution of the intracellular osmotic pressure in a developing zebrafish embryo. The pressure values are obtained from the calibration curve (Eq. 1; FIG. 2F) after measurements of the inner droplet size at each time point. Inset shows equatorial confocal sections of the inner droplet at different timepoints. Scale bar 20 μm. Error bars are measurement error for a single droplet. FIG. 10E shows time lapse of a zebrafish embryo in 2% w/w SDS solution imaged in an inverted microscope (transmitted light) and sustained on a porous membrane. Scale bar, 300 μm. FIG. 10F shows measured time evolution of the osmotic pressure during SDS treatment (2% w/w SDS). Insets show inner droplet equatorial confocal sections at different timepoints. Scale bars, 20 μm. Error bars are measurement error for a single droplet. FIG. 10G shows confocal section of a zebrafish embryo blastula at sphere stage (4 hpf) with a droplet inserted in the interstitial fluid between the cells. Scale bar, 200 μm. FIG. 10H shows close up showing the equatorial confocal section of the droplet in FIG. 10G. Scale bar, 20 μm.

FIG. 10I shows schematic representation of the droplet (oil layer 1003 and inner droplet 1004) in between adhering cells 1005 and the presence of osmolytes 1006 in the interstitial fluid. Adhesion molecules 1007 enable the cells 1005 adhere together.

FIG. 10J shows measured osmotic pressure inside blastomeres, between the cells (interstitial fluid) of the zebrafish blastula (sphere stage) and after SDS treatment. N=19, 21, 10, respectively. Osmotic pressure of E3 buffer (embryo media) with (1110) and without (1111) 2% w/w SDS, measured with a commercial osmometer. FIG. 10K shows measured osmotic pressure variation (standard deviation) of temporal readings in individual embryos and across the different embryos. N=6.

### Osmotic Pressure of Interstitial Fluid in Developing Zebrafish Embryos

Beyond intracellular osmotic pressure, the osmotic pressure of the extracellular interstitial fluid located between the cells (FIG. 10I) has also been shown to play an important role in morphogenetic events. To measure the osmotic pressure of the interstitial fluid, a double-emulsion droplet can be injected in between the cells of the zebrafish embryo blastula at sphere stage in accordance with embodiments (FIGS. 10G, H and monitored the volume of the inner droplet. The droplet is imaged every 20 min to allow proper equilibration of the droplet volume. The interstitial fluid has enough time to accommodate this change. The measured osmotic pressure of the interstitial fluid is approximately of 0.7 MPa, corresponding to an osmolality of 280 mOsm/kg on average, nearly identical (within error) to the measured intracellular value of osmotic pressure in blastomeres (FIG. 10J). This value is very close to the average osmolality of 260 mOsm/kg measured in whole zebrafish blastula explants, showing that in vivo and in situ readings are accurate.

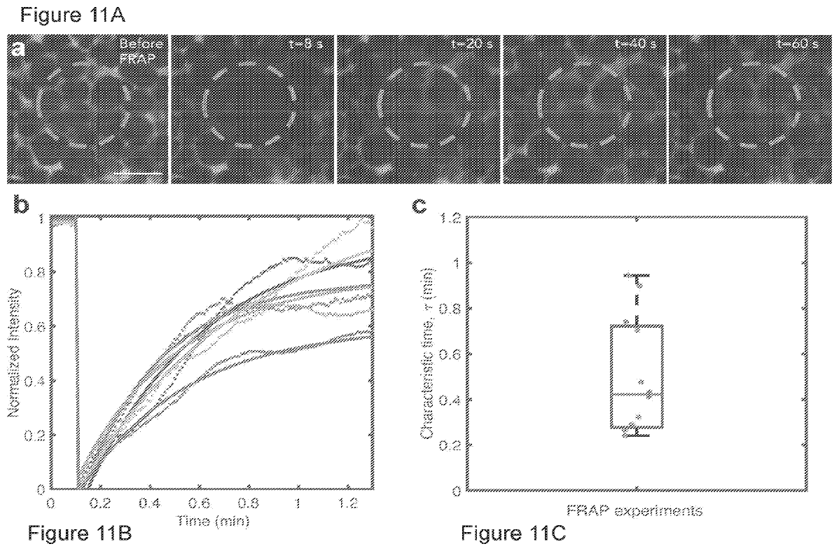

FIGS. 11A through 11C illustrate fluorescence recovery after photobleaching (FRAP) of Dextran signal in the interstitial spaces of a developing zebrafish embryo in accordance with an embodiment. FIG. 11A shows representative confocal time-lapse of an optical section of the zebrafish embryo at sphere stage during and after photobleaching, showing the dynamics of GFP-Dextran (green) in the interstitial fluid between cells. A region of interest (dashed red line) is defined to photo bleach the GFP-Dextran and monitor its recovery. Scale bar, 20 μm. FIG. 11B shows measured GFP-Dextran intensity profile (dots) as a function of time during FRAP experiment. Distinct colors indicate different experiments. Intensity fluctuations arise from cell rearrangements. Exponential fit (solid lines) are used to obtain the characteristic recovery timescale. FIG. 11C shows measured values of the characteristic fluorescence recovery timescale. Each point represents an independent experiment (N=11). Median characteristic time is 0.43 min. Boxplot show Median, 25th and 75th percentiles, whiskers extend to extreme data points.

### EXEMPLARY EMBODIMENTS

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g., amounts, temperature, etc.) but some experimental errors and deviations should be accounted for.

### Example 1: Microfluidic Device Fabrication

The microfluidic devices for producing double emulsions are made of poly(dimethyl siloxane) (PDMS Sylgard 184, Sigma Aldrich) and fabricated using soft lithography. The microfluidic devices can be made with glass or other types of polymers. As can be readily appreciated, any of a variety of material that is suitable for microfluidic devices can be utilized as appropriate to the requirements of specific applications in accordance with various embodiments of the invention. The dimensions of the device are adjusted to achieve the desired droplet sizes. Specifically, two different flow focusing devices with different dimensions of the main channel are used, namely 100 μm width and 60 μm height for the large one and 30 μm width and 30 μm height for the small one. The size of the droplets generated depended on the channel geometry. Surface activation of the PDMS devices is done with plasma treatment (Plasma Harrick PDC-32G). Then, a solution containing a cationic polymer, 2% w/w pollydiallyldimethylammonium chloride (PDADMAC, Sigma Aldrich,) and 1M NaCl(Sigma), is used to render the main channel downstream of the 3D junction hydrophilic. A solution of 2% v/v trichloro(1H, 1H,2H,2H-perfluorooctyl) silane (Sigma-Aldrich) is used to obtain fluorophilic injection channel upstream of the 3D junction.

### Example 2: Double Emulsion Droplet Composition

The inner droplet is composed of an aqueous solution containing either 5%, 10% or 20% w/w poly(ethylene glycol) (PEG, Sigma, Mw=6 kDa), corresponding to osmolalities of 16 mOsm/kg, 65.33 mOsm/kg and 420 mOsm/kg, respectively, and 0.01% w/w of mPEG-Rhodamine (Creative PEG Works, Mw=5 kDa) or mPEG-Fluorescein (Mw=5 kDa). The concentration of PEG in the inner droplet defines the internal osmotic pressure of the droplet, as PEG cannot go through the oil layer (FIG. 3). The presence of PEG also facilitates the generation of double emulsion droplets because it increases the solution viscosity. mPEG-Rhodamine is added at a much smaller concentration to enable droplet size measurements using confocal microscopy. The oil layer surrounding the inner droplet is composed of a fluorinated oil, namely hydrofluoroether (HFE) Novec™ 7700, containing a fluorinated surfactant Krytox-PEG at a 2% w/w concentration, which is a triblock surfactant that has two perfluorinated blocks that are separated by a PEG-based block. For imaging purposes, 0.025 mM of custom-made fluorinated dye F₈₆Cy5 is added to the oil phase.

### Example 3: Production of Water-In-Oil Double Emulsion Droplets

Each phase is injected in the flow focusing microfluidic device, with each flow rate (FIG. 2B; inner flow rate, Qᵢ, magenta; oil flow rate, Qₘ, cyan; outer flow rate, Qₒ, blue) being independently controlled by a different syringe pump. In addition to the phases described above for the inner droplet and the oil layer, the external aqueous phase to generate the droplet contained 10% w/w partially hydrolyzed poly(vinyl alcohol) (PVA, Mw=13-23 kDa). Water-in-oil-in-water double emulsion drops with diameters ranging from 25 to 120 μm are formed using the two flow focusing devices described above. Control over the general size of the droplet was achieved by two parameters: the type of device and outer flow rate. For droplets with initial diameter ranging from 60 to 120 μm, the large device and outer flow rate Qₒ ranging between 3000 to 6000 μL/h are used. For droplets with initial radius between 20 to 60 μm, the small device and outer flow rate Qₒ ranging between 300 to 1700 μL/h are used. To change the initial oil volume fraction,

$${V}_{oil} / {V}_{T}^{0} ,$$

a large device and kept Qₒ constant at 4500 μL/h, while the ratio Qᵢ/Qₘ is adapted from 1:1 to 8:3.

### Example 4: Osmotic Pressure Calibration

The osmolality of all aqueous solutions used for calibration and testing is measured with an osmometer. Conversion from osmolality, πₒₛₘ, to osmotic pressure, n, is done using the Van′t Hoff Law for dilute solutions, namely Π=πₒₛₘ RT, with R the gas constant and T the temperature in Kelvin.

In order to relate the internal volume of the droplet to the external pressure, the droplets have initial inner radius of 33.5±0.6 μm. Those droplets are subsequently placed into NaCl solutions with calibrated concentrations of 0.05 M, 0.1 M, 0.15 M, 0.3 M, 0.5 M and 1 M. For ionic and dilute solutions, the osmotic pressure is related to the concentration as follow. The osmolality πₒₛₘ=nφc, with n being the number of particles in which the compound dissociates, φ being the degree of dissociation of the solute and c the solute concentration. In the case if NaCl, n=2 and φ=1. Knowing n, φ, and c, for the NaCl solution, πₒₛₘ and the osmotic pressure can be obtained according to Π=πₒₛₘ RT. This provided a solution of osmotic pressure that is used to calibrate the droplets.

### Example 5: Storage and Osmotic Pressure Calibration

All droplets produced with microfluidic devices are generated and initially stored in 10% w/w PVA aqueous solution with osmolality of 79 mOsm/kg (or 200 kPa in osmotic pressure).

The osmolality of cell culture media measured in the osmometer is 839 kPa or 338 mOsm/kg. E3 embryo media is composed of NaCl (290 mg/L), KCl (13.33 mg/L), CaCl2 (4.83 mg/L), MgCl2 (81.5 mg/L) and methylene blue (1 vol %, 100 μL/L). The measured osmolality of the E3 embryo media is 11 mOsm/kg (27.3 kPa), which increased to 48 mOsm/kg (118 kPa) when SDS is added at a 2% w/w concentration.

### Example 6: Characteristic Relaxation Time

In order to characterize the characteristic timescale of pressure equilibration, the droplet volume changes can be monitored over time and fit an exponential decay to the data. The characteristic relaxation timescale is the timescale of the exponential fit.

### Example 7: Zebrafish Husbandry and Fish Lines

Zebrafish (Danio rerio) are raised and bred. A Tg(actb2: mem-NeonGreen)hm37 transgenic line is used for ubiquitous labeling of cell membrane of zebrafish embryos.

### Example 8: Injection of Double Emulsion Droplets in Zebrafish Embryos

Zebrafish embryos at 1-cell stage are chemically dechorionated by 1 mg/ml of pronase in E3 buffer. Embryos at sphere stage are dechorionated mechanically. Embryos are all microinjected with double emulsion droplet in 0.1 M KCl solution using a picolitre injector. Micropipettes for microinjection are made from microcapillaries using a Sutter P-1000 needle puller and are coated with 2% w/w PDADMAC in 1M NaCl to avoid rupture of the double emulsion droplet inside the micropipette. The diameter of the inner droplets of the double emulsions ranged between 20-35 μm. Double emulsion droplets are back-loaded into the microneedle, which tends to accumulate at the tip of the needle due to gravity. Injection pressure is tuned to achieve the injection of single droplets in the embryo.

### Example 9: Mounting and Imaging of Double Emulsion Droplets in Zebrafish Embryos

Imaging can be performed in a Zeiss LSM710 laser scanning confocal microscope. Imaging of zebrafish embryos injected with double emulsion droplets are mounted in 0.75% low-melting point agarose (Invitrogen) mixed with 25% OptiPrep™ density gradient medium in E3 buffer in a glass-bottom dish (MatTek; P35G-1.5-14-C) with two layers of silicone isolators. For SDS treatment, a 40-μm nylon mesh, which is cut out from cell strainers, is used to provide a porous seal instead of a cover slide. SDS treatment is applied at 128-cell stage of the zebrafish development and measurement of the drop size is manually performed every 15 min for 4h.

Images of early development zebrafish are taken using a 10× air objective (EC Epiplan-Neofluar 10×0.25). For measurements of volume changes in double emulsion droplets, 3D timelapses of droplets are acquired using a 40× water immersion objective (LD C-Apochromat 1.1W) at 25° C. Confocal section in z are between 5-10 μm with the 10× objective and 1-2 μm for the 40× objective

### Example 10: Imaging of Droplets for Calibration Purposes

3D confocal time lapses of droplets are acquired on a Zeiss LSM710 laser scanning confocal microscope with a 40× water immersion objective (LD C-Apochromat 1.1W) at room temperature.

### Example 11: Analysis of Inner and Outer Droplet Volumes

Matlab can be used to analyze the resulting data. First, maximum intensity projections (MIP) of the measured z-stack of a given droplet are obtained for the inner droplet at every timepoint. Individual time lapses of inner droplets' MIPs are segmented by thresholding a grayscale image with an input threshold value. Segmentation artifacts that are smaller than a critical object size is removed and binary erosion operation and binary dilation operation are applied consecutively to generate smooth droplet interface. Individual droplets are then labelled at each time point and tracked over time based on the shortest distance criterion between consecutive time points. For each droplet identified in the segmented image, the droplet area A is computed by counting number of pixels. Since the inner droplets maintained spherical shape, the inner droplet volume is obtained from

$${V}_{I} ( t ) = \frac{4}{3} {π ( \sqrt{\frac{A ( t )}{π}} )}^{3} .$$

### Example 12: Fluorescence Recovery after Photobleaching

A solution of Dextran-Alexa Fluor 488 (10,000 MW) in DI water at a concentration of 10 mg/mL is prepared, and approximately 0.5 nL of this solution is injected in the interstitial fluid of a zebrafish embryo at the sphere stage following the same protocol as for injection of droplets (see above). After 20 min, the zebrafish embryos are mounted for imaging as described above. Imaging was done as described above and using a 25× water objective. To measure the fluorescence recovery after photobleaching, a Region of Interest (ROI) of 30 μm by 30 μm is defined and the fluorescence signal within this region was photobleached by illuminating it with a 488 nm laser (80% laser power; 10 frames). Fluorescence intensity was then monitored in the defined ROI for 15 min after photobleaching. The average intensity in the ROI is measured and fitted with a single exponential function to obtain the recovery timescale.

### Example 13: Reproducibility

The characteristic reduction of volume and subsequent stabilization of the double emulsion droplets submitted to osmotic pressure is independently observed 86 times (FIG. 2D). Observations reported in FIGS. 2B and 2C are reproducible and observed 20 times. Observations of droplets in zebrafish embryos at different stages (FIGS. 10A and 4G) are reproducible and observed 50 times.

### Examples

Example 1: An osmotic pressure sensor comprising: a double emulsion droplet comprising: an oil droplet with a first diameter; wherein the oil droplet has an inner surface and an outer surface and the inner and the outer surfaces are modified with at least one surfactant to facilitate water transport through the oil droplet; and at least one aqueous droplet enclosed in the oil droplet; wherein the at least one aqueous droplet has a second diameter less than the first diameter; wherein the at least one aqueous droplet comprises an inner osmolyte and an environment outside the double emulsion droplet comprises an outer osmolyte; wherein a concentration difference between the outer osmolyte and the inner osmolyte drives water transport through the oil droplet such that the double emulsion droplet either shrinks or expands in volume; and wherein a volume change in the double emulsion droplet measures the osmotic pressure.
Example 2: The example osmotic pressure sensor of example 1, wherein the first diameter is less than or equal to 160 microns.
Example 3: The osmotic pressure sensor of example 1 or 2, wherein the inner osmolyte has a molecular weight of less than 6,000 Dalton.
Example 4: The osmotic pressure sensor of example 1, or 2, or 3, wherein the oil droplet comprises an oil selected from the group consisting of: a fluorocarbon oil, a silicone oil, and a biocompatible oil.
Example 5: The osmotic pressure sensor of any one of examples 1 to 4, wherein the surfactant comprises a molecular component with at least one hydrophilic part and at least one hydrophobic part, lipids, sodium dodecyl sulfate, sodium lauryl sulfate, poly(N-isopropylacrylamide), or a fluorinated surfactant.
Example 6: The osmotic pressure sensor of any one of examples 1 to 5, wherein the inner osmolyte comprises polyethylene glycol or Dextran; the oil droplet comprises a fluorocarbon oil, a silicone oil, or a biocompatible oil; and the at least one surfactant comprises Krytox™-PEG (600), Krytox™, or perfluoro-15-crown-5-ether.
Example 7: The osmotic pressure sensor of any one of examples 1 to 6, wherein the at least one aqueous droplet further comprises a color dye or a fluorescent color dye.
Example 8: The osmotic pressure sensor of any one of examples 1 to 7, wherein the oil droplet further comprises a color dye or a fluorescent color dye.
Example 9: The osmotic pressure sensor of any one of examples 1 to 8, wherein the osmotic pressure sensor is configured to measure the osmotic pressure in vitro, in vivo or in situ.
Example 10: A method for measuring an osmotic pressure, comprising: obtaining a double emulsion droplet, wherein the droplet comprises: an oil droplet with a first diameter; wherein the oil droplet has an inner surface and an outer surface and the inner and the outer surfaces are modified with at least one surfactant to facilitate water transport through the oil droplet; and at least one aqueous droplet enclosed in the oil droplet; wherein the at least one aqueous droplet has a second diameter less than the first diameter; wherein the at least one aqueous droplet comprises an inner osmolyte; and wherein an osmolyte concentration difference drives water transport through the oil droplet such that the double emulsion droplet either shrinks or expands in volume; calibrating a relationship between a volume change of the double emulsion droplet and an external osmotic pressure in a reference solution; placing the double emulsion droplet in an environment comprising an outer osmolyte; monitoring a volume change of the double emulsion droplet in the environment; and obtaining the osmotic pressure of the environment using the calibrated relationship.
Example 11: The method of example 10, further comprising using a microscope to monitor the volume change of the double emulsion droplet in the environment.
Example 12: The method of example 10 or 11, further comprising placing the double emulsion droplet in the environment using a microneedle.
Example 13: The method of example 10, 11, or 12, wherein the first diameter is less than or equal to 160 microns.
Example 14: The method of any one of examples 10 to 13, wherein the inner osmolyte has a molecular weight of less than 6,000 Dalton.
Example 15: The method of any one of examples 10 to 14, wherein the oil droplet comprises an oil selected from the group consisting of: a fluorocarbon oil, a silicone oil, and a biocompatible oil.
Example 16: The method of any one of examples 10 to 15, wherein the surfactant comprises a molecular component with at least one hydrophilic part and at least one hydrophobic part, lipids, sodium dodecyl sulfate, sodium lauryl sulfate, poly(N-isopropylacrylamide), or a fluorinated surfactant.
Example 17: The method of any one of examples 10 to 16, wherein the inner osmolyte comprises polyethylene glycol or Dextran; the oil droplet comprises a fluorocarbon oil, a silicone oil, and a biocompatible oil; and the at least one surfactant comprises Krytox™-PEG (600), Krytox™, or perfluoro-15-crown-5-ether.
Example 18: The method of any one of examples 10 to 17, wherein the at least one aqueous droplet further comprises a color dye or a fluorescent color dye.
Example 19: The method of any one of examples 10 to 18, wherein the oil droplet further comprises a color dye or a fluorescent color dye.
Example 20: The method of any one of examples 10 to 19, wherein the environment is an in vitro environment, an in vivo environment, or an in situ environment.

### Doctrine of Equivalents

As can be inferred from the above discussion, the above-mentioned concepts can be implemented in a variety of arrangements in accordance with embodiments of the invention. Accordingly, although the present invention has been described in certain specific aspects, many additional modifications and variations would be apparent to those skilled in the art. It is therefore to be understood that the present invention may be practiced otherwise than specifically described. Thus, embodiments of the present invention should be considered in all respects as illustrative and not restrictive.

As used herein, the singular terms “a,” “an,” and “the” may include plural referents unless the context clearly dictates otherwise. Reference to an object in the singular is not intended to mean “one and only one” unless explicitly so stated, but rather “one or more.”

As used herein, the terms “approximately,” and “about” are used to describe and account for small variations. When used in conjunction with an event or circumstance, the terms can refer to instances in which the event or circumstance occurs precisely as well as instances in which the event or circumstance occurs to a close approximation. When used in conjunction with a numerical value, the terms can refer to a range of variation of less than or equal to +10% of that numerical value, such as less than or equal to +5%, less than or equal to +4%, less than or equal to +3%, less than or equal to +2%, less than or equal to ±1%, less than or equal to ±0.5%, less than or equal to ±0.1%, or less than or equal to ±0.05%.

Additionally, amounts, ratios, and other numerical values may sometimes be presented herein in a range format. It is to be understood that such range format is used for convenience and brevity and should be understood flexibly to include numerical values explicitly specified as limits of a range, but also to include all individual numerical values or sub-ranges encompassed within that range as if each numerical value and sub-range is explicitly specified. For example, a ratio in the range of about 1 to about 200 should be understood to include the explicitly recited limits of about 1 and about 200, but also to include individual ratios such as about 2, about 3, and about 4, and sub-ranges such as about 10 to about 50, about 20 to about 100, and so forth.

1. An osmotic pressure sensor comprising:
a double emulsion droplet comprising:
an oil droplet with a first diameter; wherein the oil droplet has an inner surface and an outer surface and the inner and the outer surfaces are modified with at least one surfactant to facilitate water transport through the oil droplet; and
at least one aqueous droplet enclosed in the oil droplet; wherein the at least one aqueous droplet has a second diameter less than the first diameter;
wherein the at least one aqueous droplet comprises an inner osmolyte and an environment outside the double emulsion droplet comprises an outer osmolyte;
wherein a concentration difference between the outer osmolyte and the inner osmolyte drives water transport through the oil droplet such that the double emulsion droplet either shrinks or expands in volume; and
wherein a volume change in the double emulsion droplet measures the osmotic pressure.

2. The osmotic pressure sensor of claim 1, wherein the first diameter is less than or equal to 160 microns.

3. The osmotic pressure sensor of claim 1, wherein the inner osmolyte has a molecular weight of less than 6,000 Dalton.

4. The osmotic pressure sensor of claim 1, wherein the oil droplet comprises an oil selected from the group consisting of: a fluorocarbon oil, a silicone oil, and a biocompatible oil.

5. The osmotic pressure sensor of claim 1, wherein the surfactant comprises a molecular component with at least one hydrophilic part and at least one hydrophobic part, lipids, sodium dodecyl sulfate, sodium lauryl sulfate, poly(N-isopropylacrylamide), or a fluorinated surfactant.

6. The osmotic pressure sensor of claim 1, wherein the inner osmolyte comprises polyethylene glycol or Dextran; the oil droplet comprises a fluorocarbon oil, a silicone oil, or a biocompatible oil; and the at least one surfactant comprises Krytox™ PEG (600), Krytox™, or perfluoro-15-crown-5-ether.

7. The osmotic pressure sensor of claim 1, wherein the at least one aqueous droplet further comprises a color dye or a fluorescent color dye.

8. The osmotic pressure sensor of claim 1, wherein the oil droplet further comprises a color dye or a fluorescent color dye.

9. The osmotic pressure sensor of claim 1, wherein the osmotic pressure sensor is configured to measure the osmotic pressure in vitro, in vivo or in situ.

10. A method for measuring an osmotic pressure, comprising:
obtaining a double emulsion droplet, wherein the droplet comprises:
an oil droplet with a first diameter; wherein the oil droplet has an inner surface and an outer surface and the inner and the outer surfaces are modified with at least one surfactant to facilitate water transport through the oil droplet; and
at least one aqueous droplet enclosed in the oil droplet; wherein the at least one aqueous droplet has a second diameter less than the first diameter;
wherein the at least one aqueous droplet comprises an inner osmolyte; and
wherein an osmolyte concentration difference drives water transport through the oil droplet such that the double emulsion droplet either shrinks or expands in volume;
calibrating a relationship between a volume change of the double emulsion droplet and an external osmotic pressure in a reference solution;
placing the double emulsion droplet in an environment comprising an outer osmolyte;
monitoring a volume change of the double emulsion droplet in the environment; and
obtaining the osmotic pressure of the environment using the calibrated relationship.

11. The method of claim 10, further comprising using a microscope to monitor the volume change of the double emulsion droplet in the environment.

12. The method of claim 10, further comprising placing the double emulsion droplet in the environment using a microneedle.

13. The method of claim 10, wherein the first diameter is less than or equal to 160 microns.

14. The method of claim 10, wherein the inner osmolyte has a molecular weight of less than 6,000 Dalton.

15. The method of claim 10, wherein the oil droplet comprises an oil selected from the group consisting of: a fluorocarbon oil, a silicone oil, and a biocompatible oil.

16. The method of claim 10, wherein the surfactant comprises a molecular component with at least one hydrophilic part and at least one hydrophobic part, lipids, sodium dodecyl sulfate, sodium lauryl sulfate, poly(N-isopropylacrylamide), or a fluorinated surfactant.

17. The method of claim 10, wherein the inner osmolyte comprises polyethylene glycol or Dextran; the oil droplet comprises a fluorocarbon oil, a silicone oil, and a biocompatible oil; and the at least one surfactant comprises Krytox™-PEG (600), Krytox™, or perfluoro-15-crown-5-ether.

18. The method of claim 10, wherein the at least one aqueous droplet further comprises a color dye or a fluorescent color dye.

19. The method of claim 10, wherein the oil droplet further comprises a color dye or a fluorescent color dye.

20. The method of claim 10, wherein the environment is an in vitro environment, an in vivo environment, or an in situ environment.
